(19) United States
(12) Patent Application Publication
(10) Pub. No.: US 2004/0052836 A1
(43) Pub. Date: Mar. 18, 2004

(54) Pharmaceutical compositions containing at least one stable liposphere having an improved shelf life

(76) Inventors: Luk Chiu Li, Lake Forest, IL (US); Hung-Chih Chang, Carmel, IN (US); Siriporn Toongsuwan, Gurnee, IL (US); Lihua Zhu, Gurnee, IL (US); Bryan K. Erickson, Pleasant Prairie, WI (US)

Correspondence Address: STEVEN F. WEINSTOCK, ABBOTT LABORATORIES, 100 ABBOTT PARK ROAD, DEPT. 377/AP6A, ABBOTT PARK, IL, 60064-6008, US

(21) Appl. No.: 10/280,221

(22) Filed: Oct. 25, 2002

Related U.S. Application Data
(60) Provisional application No. 60/410,361, filed on Sep. 13, 2002.

(51) Int. Cl. A61K 9/127

(52) U.S. Cl. 424/450

(57) ABSTRACT

The present invention relates to pharmaceutical compositions that contain at least one liposphere that is being used as a drug delivery vehicles to deliver at least one compound to a patient in need of treatment.

This application claims priority to the provisional application Serial No. 60/410,361 filed on Sep. 13, 2002.

### FIELD OF THE INVENTION

The present invention relates to pharmaceutical compositions containing at least one stable liposphere that can be used as a controlled delivery system to administer one or more compounds, such as drugs, to a patient in need of treatment. The present invention also relates to methods for making such lipospheres.

### BACKGROUND OF THE INVENTION

Lipospheres are solid, water-insoluble microparticles having a solid hydrophobic core and a layer of a phospholipid embedded on the surface of the core. The internal hydrophobic core contains a compound that is dissolved or dispersed in the solid-fat matrix. Lipospheres can be used for the controlled delivery of various types of drugs, including anti-inflammatory compounds, local anesthetics, antibiotics, cancer agents, etc. Lipospheres can also be used as carriers of vaccines and adjuvants.

Liposphere formulations can be prepared by using a solvent or melt process. In the solvent process, the active agent, the solid carrier and phospholipid are dissolved in an organic solvent such as acetone, ethyl acetate, ethanol, or dichloromethane. The solvent is then evaporated and the resulting solid mixed with a warm buffer solution and mixed until a homogenous dispersion of lipospheres is obtained.

Alternatively, in the melt method, the active agent is dissolved or dispersed in the melted solid carrier. The hot mixture is then homogenized for about 2-5 minutes using a homogenizer or ultrasound probe, after which a uniform emulsion is obtained. The milky formulation is then rapidly cooled down to about 20° C. by immersing the container in an acetone-dry ice bath while homogenization is continued to yield a uniform dispersion of lipospheres. See Microencapsulation—Methods and Industrial Applications, edited by Simon Benita, Marcel Dekker, Inc. 1996, p. 379.

Traditionally, natural phospholipids, such as, egg phosphatidylcholine, soy phosphatidylcholine, etc. have been used in the preparation of lipospheres. However, lipospheres made with natural phospholipids exhibit physical stability problems and have a relatively short shelf life. Therefore, there is a need in the art for new and improved lipospheres that are stable and exhibit a long shelf life.

### SUMMARY OF THE INVENTION

In one embodiment, the present invention relates to pharmaceutical compositions. The pharmaceutical compositions of the present invention comprise lipospheres having an average particle size of at least about 3 microns and not greater than about 450 microns. The lipospheres comprise (1) at least one glyceride, at least one synthetic phospholipid and at least one compound; and/or (2) at least one glyceride, at least one phospholipid (natural or synthetic), at least one polymer and at least one compound.

The glyceride in the lipospheres can be a monoglyceride, diglyceride or triglyceride. Examples of triglycerides that can be used include, but are not limited to, trilaurin, tricaprin, triarachidin or trimyristin. Synthetic phospholipids that can be used in the lipospheres include, but are not limited to, dimyristoylphosphatidylcholine, dipalmitoylphosphatidylcholine, distearoylphosphatidylcholine, dilauroylphosphatidylcholine, or diarachidoylphosphatidylcholine. Natural phospholipids that can be used include, but are not limited to, egg phosphatidylcholine or soy phosphatidylcholine.

With respect to the polymer, any type of polymer can be used in the lipospheres. For example, the polymer can be a cellulose polymer, such as, but not limited to, carboxymethylcellulose salt, hydroxypropylmethylcellulose or methylcellulose. The compound contained in the liposphere can be a drug such as antianginas, antiarrhythmics, antiasthmatic agents, antibiotics, antidiabetics, antifungals, antihistamines, antihypertensives, antiparasitics, antineoplastics, antitumor drugs, antivirals, cardiac glycosides, herbicides hormones, immunomodulators, monoclonal antibodies, neurotransitters, nucleic acid proteins, radio contrast agents, radionuclides, sedatives, analgesics, steroids, tranquilizers, vaccines, vasopressors, anesthetics, peptides and the like. Prodrugs that undergo conversion to an active drug upon local interactions with the intracellular medium, cells or tissues can also be used in the lipospheres. For example, if the compound is a drug, such as a local anesthetic, bupivacaine can be used.

Liposphere(s) contained in the pharmaceutical compositions of the present invention can contain varying amounts of glyceride(s), phospholipid(s) (natural or synthetic), polymer(s) (if present) and compound(s) depending upon (1) the type of phospholipid used (natural versus synthetic) and/or (2) the amount of compound(s) to be delivered. For example, for lipospheres that contain at least one glyceride(s), at least one synthetic phospholipid and at least one compound(s), if the compound is present in the amount of from about 2.0% to less than about 2.5% weight to volume, then the amount of glyceride(s) present is in the amount of from about 3.0% to about 9.0% weight to volume and the amount of synthetic phospholipid(s) present is in the amount of from about 0.5% to about 3.0% weight to volume. Alternatively, if the compound is present in the amount of from about 2.5% to about 3.0% weight to volume, then the amount of glyceride(s) present is in the amount of from about 3.0% to about 9.0% weight to volume and the synthetic phospholipid(s) is present in the amount of from about 0.75% to about 3.0% weight to volume with the following provisos (1) that if the glyceride is present in the amount of about 3.0% weight to volume, then the synthetic phospholipid(s) is present in the amount of more than about 0.75% by weight to volume; or (2) that if the glyceride(s) is present in the amount of about 9.0% by weight to volume then said synthetic phospholipid(s) must be present in the amount of less than about 3.0% weight to volume. Lipospheres having any of the above-described formulations have a gel time of at least 24 hours at room temperature.

For lipospheres that contain at least one glyceride(s), at least one phospholipid, at least one polymer(s) and at least one compound(s), if a natural phospholipid is used and if the compound is present in the amount of from about 2.0% to 3.0% weight to volume, then the amount of glyceride(s) present is in the amount of from about 3.0% to about 9.0% to weight to volume, the amount of natural phospholipid(s) present is in the amount of from about 0.5% to less than about 3.0% weight to volume and the amount of polymer(s) present is in the amount of from about 0.1% to about 0.5% weight to volume. Lipospheres having such a formulation have a gel time of 24 hours at room temperature.

If a synthetic phospholipid is used and if the compound is present in the amount of from about 2.0% to less than about 2.5% weight to volume, then the amount of glyceride(s) present is in the amount of from about 3.0% to about 9.0% weight to volume, the amount of synthetic phospholipid(s) present is in the amount of from about 0.5% to about 3.0% weight to volume and the amount of polymer(s) present is in the amount of from about 0.1% to about 0.5% weight to volume. Lipospheres having such a formulation have a gel time of at least 15 days at room temperature. Alternatively, if the compound is present in the amount of from about 2.5% to about 3.0% weight to volume, then the amount of glyceride(s) present is in the amount of from about 3.0% to about 9.0% weight to volume, the amount of polymer(s) present is in the amount of from about 0.1% to about 0.5% weight to volume and the synthetic phospholipid(s) is present in the amount of from about 0.75% to about 3.0% weight to volume with the proviso that if the glyceride(s) is present in the amount of about 3.0% by weight to volume then said synthetic phospholipid(s) must be present in the amount of more than about 0.75% weight to volume. Lipospheres having such a formulation have a gel time of at least 30 days at room temperature.

In yet another embodiment, the present invention relates to a method of preparing lipospheres. The method comprises the following steps. First, at least one glyceride(s), at least one phospholipid(s) (synthetic or natural), at least one compound(s) and a dispersion medium is provided for use in the method. Next, the glyercide, phospholipid, compound and dispersion medium are placed in a reaction vessel and heated until each of the ingredients has melted to form a mixture. Next, the mixture is homogenized to form a dispersion. Next, the mixture is cooled to a temperature of from about 0° C. to about 10° C. while the mixture is stirred until a suspension of lipospheres having an average particle size of at least about 3 microns and not greater than about 450 microns is formed. Optionally, at least one polymer(s) can be provided along with the glyceride(s), phospholipid(s) and compound in the first step. Alternatively, at least one polymer(s) can be added to the suspension of lipospheres formed as a result of the cooling.

The glyceride employed in the method can be a monoglyceride, diglyceride or triglyceride. Examples of triglycerides that can be used include, but are not limited to, trilaurin, tricaprin, triarachidin or trimyristin. Synthetic phospholipids that can be used in the method include, but are not limited to, dimyristoylphosphatidylcholine, dipalmitoylphosphatidylcholine, distearoylphosphatidylcholine, dilauroylphosphatidylcholine, or diarachidoylphosphatidylcholine. Natural phospholipids that can be used in the method include, but are not limited to, egg phosphatidylcholine or soy phosphatidylcholine.

With respect to the polymer, any type of polymer can be used in the method. For example, the polymer can be gelatin, polyvinyl pyroolidone (PVP), polyethylene glycol (PEG), alginate, and/or a cellulose polymer, such as, but not limited to, carboxymethylcellulose salt, hydroxypropylmethylcellulose or methylcellulose. The compound used in the method can be a drug such as antianginas, antiarrhythmics, antiasthmatic agents, antibiotics, antidiabetics, antifungals, antihistamines, antihypertensives, antiparasitics, antineoplastics, antitumor drugs, antivirals, cardiac glycosides, herbicides, hormones, immunomodulators, monoclonal antibodies, neurotransitters, nucleic acid proteins, radio contrast agents, radionuclides, sedatives, analgesics, steroids, tranquilizers, vaccines, vasopressors, anesthetics, peptides and the like. Prodrugs that undergo conversion to an active drug upon local interactions with the intracellular medium, cells or tissues can also be used in the lipospheres. For example, if the compound is a drug, such as a local anesthetic, bupivacaine can be used.

In yet another embodiment, the present invention relates to a method of inducing or maintaining local anesthesia in a patient. The method involves the step of administering to a patient an effective amount of any one of the previously described pharmaceutical compositions to induce local anesthesia. The method can also employ the use of a pharmaceutically acceptable excipient with the pharmaceutical composition.

### BRIEF DESCRIPTION OF THE FIGURES

FIG. 1 shows the difference between a free flowing sample (upper tube) of lipospheres and a gelled sample of lipospheres (lower tube).

FIG. 2 shows the results of a rat thermal paw flick anesthesia study using bupivacaine.

FIG. 3 shows the results of a guinea pig wheal anesthesia study using bupivacaine.

### DEFINITIONS

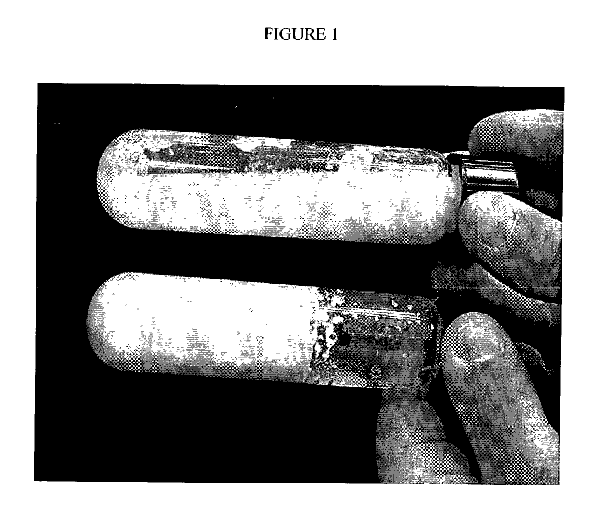

As used herein, the term “gellation time” or “gel time”, as used interchangeably herein, refers to the amount of time it takes for a dispersion of lipospheres to (1) exhibit resistance to flowing freely when tilted in a container of lipospheres (See FIG. 1); and/or (2) physically appear to have solidified, coalesced, coagulated and/or separated.

As used herein, the term “lyophilize” or “freeze drying” refers to the preparation of a liposphere dispersion in dry form by rapid freezing and dehydration in a frozen state. Lyophilization takes place at a temperature that results in the freezing of the liposphere dispersion. The process takes place under a vacuum at a pressure sufficient to maintain frozen product.

As used herein, the term “melting point” means the temperature at which solid (such as, but not limited to, a compound) becomes a liquid.

As used herein, the term “patient” refers to animals, including mammals, specifically, humans.

As used herein the term “polymer” refers to molecules formed from the chemical union of two or more repeating units. The term “polymer” includes homopolymers, copolymers, block copolymers, etc. The polymers that can be used in the present invention can be synthetic, naturally-occurring or semi-synthetic. The polymer can also be biodegradable polymers, which are polymers that can be degraded to a low molecular weight and may or may not be eliminated from a living organism.

As used herein, the term “weight to volume” means the number of grams (g) per 100 milliliters (mL) of formulation.

### DETAILED DESCRIPTION OF THE PRESENT INVENTION

Pharmaceutical Compositions Containing Lipospheres made from Synthetic Phospholipids Having an Improved Gellation Time

In one embodiment, the present invention relates to a pharmaceutical composition containing lipospheres wherein at least one liposphere in the pharmaceutical composition comprises at least one glyceride, at least one synthetic phospholipid and at least one compound and has a particle size of at least about 3 microns in diameter and not greater than about 450 microns in diameter. The inventors of the present invention have found that when at least one synthetic phospholipid is used to make the liposphere used in the pharmaceutical composition of the present invention that the lipospheres exhibit improved stability and have a longer gellation time than lipospheres known in the art. More specifically, the lipospheres of the present invention exhibit a longer shelf life and are less expensive to make when compared to lipospheres known in the art that are made from natural phospholipids.

The lipospheres contained in the pharmaceutical composition of the present invention are made from and contain at least one glyceride. The glyceride can be at least one monoglyceride, diglyceride or triglyceride or combinations thereof and can have varying degrees of unsaturation. Examples of monoglycerides that can be used include, but are not limited to, monocaprin (C-10), monolaurin (C-12), monomyristin (C-14), monopalmitin (C-16) and monostearin (C-18). Examples of diglycerides that can be used include, but are not limited to, dicaprin (C-10), dilaurin (C-12), dimyristin (C-14), dipalmitin (C-16), distearin (C-18) and diarachidin (C-20). Examples of triglycerides that can be used include, but are not limited to, tricaprin (C-10), trilaurin (C12), trimyristin (C-14), tripalmitin (C-16), tristearin (C-18) and triarachidin (C-20). The glyceride(s) used in the present invention are available from commercial suppliers such as Condea Vista (Houston, Tex.) and Sigma-Aldrich Corp. (St. Louis, Mo.). The glyceride(s) is present in the liposphere in the amount of from about 3.0% to about 9.0% weight to volume.

In addition to at least one glyceride(s), the lipospheres are made from and contain at least one synthetic phospholipid. Examples of synthetic phospholipids that can be used include dimyristoylphosphatidylcholine, dipalmitoylphosphatidylcholine, dilauroylphosphatidylcholine, distearoylphosphatidylcholine, or diarchidoylphosphatidylcholine. Such synthetic phospholipids are available from commercial suppliers such as Avanti Polar Lipids, Inc. (Alabaster, Ala.) and Genzyme Pharmaceuticals (Cambridge, Mass.). The synthetic phospholipid is present in the liposphere in the amount of from about 0.5% to about 3.0% weight to volume.

The liposphere also is made from and contains at least one compound. The compound is present in the liposphere in the amount of from about 2.0% to about 3.0% weight to volume. The compound contained in the liposphere can be a drug such as antianginas, antiarrhythmics, antiasthmatic agents, antibiotics, antidiabetics, antifungals, antihistamines, antihypertensives, antiparasitics, antineoplastics, antitumor drugs, antivirals, cardiac glycosides, herbicides, hormones, immunomodulators, monoclonal antibodies, neurotransitters, nucleic acid proteins, radio contrast agents, radionuclides, sedatives, analgesics, steroids, tranquilizers, vaccines, vasopressors, anesthetics, peptides and the like. Prodrugs that undergo conversion to an active drug upon local interactions with the intracellular medium, cells or tissues can also be used in the lipospheres. The compound used in the liposphere can have a melting point of about 90° C. to about 115° C.

If the compound is a drug, such as an anesthetic, the anesthetic can be a local anesthetic. Examples of local anesthetics include, but are not limited to, bupivacaine, ropivacaine, dibucaine, procaine, chloroprocaine, prilocaine, mepivacaine, etidocaine, tetracaine, lidocaine, and xylocaine, as well as anesthetically active derivatives, analogs and mixtures thereof.

The liposphere(s) contained in the pharmaceutical composition of the present invention can contain varying amounts of glyceride(s), synthetic phospholipid(s) and compound(s) depending upon the amount of compound(s) to be delivered. For example, when the compound, such as bupivacaine, is present in the amount of from about 2.0% to less than about 2.5% weight to volume, then the amount of glyceride(s) present is in the amount of from about 3.0% to about 9.0% to weight to volume and the amount of synthetic phospholipid(s) present is in the amount of from about 0.5% to about 3.0% weight to volume. Alternatively, if the compound is present in the amount of from about 2.5% to about 3.0% weight to volume, then the amount of glyceride(s) present is in the amount of from about 3.0% to about 9.0% weight to volume and the synthetic phospholipid(s) is present in the amount of from about 0.75% to about 3.0% weight to volume with the following provisos (1) that if the glyceride is present in the amount of about 3.0% weight to volume, then the synthetic phospholipid(s) is present in the amount of more than about 0.75% by weight to volume; or (2) that if the glyceride(s) is present in the amount of about 9.0% by weight to volume then said synthetic phospholipid(s) must be present in the amount of less than about 3.0% weight to volume.

In addition to the glyceride(s), synthetic phospholipid(s) and compound(s), the lipospheres can also optionally contain one or more preservatives, antioxidants, buffers or surfactants. More specifically, the liposphere(s) can contain from about 0.01% to about 2.0% weight to volume of a preservative to prevent microbial growth, such as, but not limited to, methylparaben, propylparaben, or benzyl alcohol. The liposphere(s) can also contain from about 0.0001% to about 1% weight to volume of an antioxidant to prevent oxidation of the formulation. Examples of antioxidants that can be used include, but are not limited to, butylated hydroxyanisole (BHA), butylated hydroxytoluene (BHT), ascorbic acid, and sodium metabisulfite. The liposphere can also contain a buffer to control the pH of the liposphere. Examples of buffers that can be used include, but are not limited to, a phosphate buffer (pH of about 5 to about 8). Suitable preservatives, antioxidants and buffers for use in the lipospheres of the present invention can be purchased from Sigma-Aldrich Corp. (St. Louis, Mo.) and J. T. Baker (Phillipsburg, N.J.).

In comparison to lipospheres known in the art that contain natural phospholipids, the inventors have discovered that the presence of the synthetic phospholipid(s) in the lipospheres imparts improved stability to the lipospheres and results in the lipospheres having a gellation time of at least about 24 hours at room temperature. For example, as shown in Example 2, lipospheres containing synthetic phospholipids made as described herein exhibited a gellation time of 58 days at room temperature. In contrast, as shown in Example 3, lipospheres made using a natural phospholipid(s) (such as egg phosphatidylcholine) exhibited a gellation time of 2 hours at room temperature.

Pharmaceutical Compositions Containing at Least One Polymer Containing Lipospheres Having Improved Gellation Time

In a second embodiment, the present invention relates to a pharmaceutical composition containing lipospheres wherein at least one liposphere in said pharmaceutical composition comprises at least one glyceride, at least one phospholipid (either a natural phospholipid or a synthetic phospholipid), at least one polymer(s) and at least one compound and has a particle size of at least about 3 microns in diameter and not greater than about 450 microns in diameter. The inventors of the present invention have found that when at least one polymer(s) is used to make the liposphere used in the pharmaceutical composition of the present invention that the lipospheres exhibit improved stability and have a longer gellation time than lipospheres known in the art. More specifically, the lipospheres of the present invention exhibit a longer shelf life when compared to lipospheres known in the art.

The lipospheres contained in the pharmaceutical composition of the present invention are made from and contain at least one glyceride. The glyceride can be at least one monoglyceride, diglyceride or triglyceride or combinations thereof and can have varying degrees of unsaturation. Examples of monoglycerides that can be used include, but are not limited to, monocaprin (C-10), monolaurin (C-12), monomyristin (C-14), monopalmitin (C-16) and monostearin (C-18). Examples of diglycerides that can be used include, but are not limited to, dicaprin (C-10), dilaurin (C-12), dimyristin (C-14), dipalmitin (C-16), distearin (C-18) and diarachidin (C-20). Examples of triglycerides that can be used include, but are not limited to, tricaprin (C-10), trilaurin (C-12), trimyristin (C-14), tripalmitin (C-16), tristearin (C-18) and triarachidin (C-20). The glyceride(s) used in the present invention are available from commercial suppliers such as Condea Vista (Houston, Tex.) and Sigma-Aldrich Corp. (St. Louis, Mo.). The glyceride(s) is present in the liposphere in the amount of from about 3.0% to about 9.0% weight to volume.

In addition to at least one glyceride(s), the lipospheres are made from and contain at least one phospholipid. The phospholipid that can be used can be of a natural or synthetic origin. Naturally occurring phospholipids can be obtained from natural lecithins that are derived from a variety of sources such as egg, bovine heart and soya bean. Examples of natural phospholipids that can be used include egg phosphatidylcholine and soy phosphatidylcholine. These natural phospholipids are available from commercial suppliers such as Avanti Polar Lipids, Inc. (Alabaster, Ala.). The natural phospholipid present in the liposphere in the amount of from about 0.5% to about less than about 3.0% weight to volume. Examples of synthetic phospholipids that can be used include dimyristoylphosphatidylcholine, dipalmitoylphosphatidylcholine, distearoylphosphatidylcholine, dilauroylphosphatidylcholine or diarchidoylphosphatidylcholine. Such synthetic phospholipids are available from commercial suppliers such as Avanti Polar Lipids, Inc. (Alabaster, Ala.) and Genzyme Pharmaceuticals (Cambridge, Mass.). The synthetic phospholipid is present in the liposphere in the amount of from about 0.5% to about 3.0% weight to volume.

In addition to at least one glyceride(s) and at least one phospholipid(s), the liposphere(s) of the present invention also is made from and contain at least one polymer(s). Any type of polymer, such as a homopolymer, copolymer, block copolymer, cellulose polymer, etc. can be used in the present invention. For example, homopolymers made from the following monomer units can be used: hydroxyl carboxylic acids such as alpha-hydroxy carboxylic acids including, but not limited to, lactic acid, glycolic acid, lactide (intermolecularly esterified dilactic acid), and glycolide (intermolecularly esterified diglycolic acid); beta-hydroxy carboxylic acids including, but not limited to, β-methyl-α-propiolactone, λ-hydroxy carboxylic acids, δ-hydroxy carboxylic acids, and ε-hydroxy carboxylic acids including ε-hydroxy caproic acid; lactones such as, but not limited to, β-lactones, γ-lactones, ε-lactones, such as, but not limited to, valerolactone and ε-caprolactone; benzyl ester-protected lactones such as benzyl malolactone; lactams such as, but not limited to, β-lactams, γ-lactams, δ-lactams, ε-lactones, thilactones such as 1,4-dithiane-2,5-dione, dioxanones, unfunctionalized cyclic carbonates, such as, but not limited to, trimethylene carbonate, alkyl substituted trimethylene carbonates, and spiro-bis-dimethylene carbonate (2,4,7,9-tetraoxa-spiro[5.5]undecan-3,8-dione), anhydrides, substituted N-carboxy anhydrides, propylene fumarates, orthoesters, phosphate esters, phosphazenes, alkylcyanoacrylates, amino acids, polyhydroxybutyrates, and substituted variations on the above-identified monomers.

The above listed monomers can be used to produce homopolymers such as, but not limited to, polylactide, polyglycolide, poly(p-dioxanone), polypropylenefumarate, polyorthoesters, polyphosphate esters, polyanhydrides, polyphosphazenes, polyalkylcyanoacrylates, polypeptides, or genetically engineered polymers.

Other polymers that can be used include gelatin, polyvinyl pyrrolidone (PVP), polyethylene glycol (PEG) and alginate.

Examples of cellulose polymers that can be used include, but are not limited to, carboxymethylcellulose salt, hydroxypropylmethylcellulose or methylcellulose, can also be used in the lipospheres of the present invention.

Examples of copolymers that can be used include, but are not limited to, poly(lactide-glycolide), poly(p-dioxanone-lactide), poly(p-dioxanone-glycolide), poly(pdioxanone-lactide-glycolide), poly(p-dioxanone-caprolactone), poly(p-dioxanonealkylene carbonate), poly(p-dioxanone-alkylene oxide), poly(p-dioxanone-carbonateglycolide), poly(p-dioxanone-carbonate), poly(caprolactone-lactide), poly (caprolactoneglycolide), poly(hydroxyalkanoate), poly(propylenefumarate), poly(orthoesters), poly(ether-ester), poly(ester-amide), poly(ester-urethane), polyphosphate esters, polyanhydrides, poly(ester-anhydride), polyphospazenes, polypeptides or genetically engineered polymers.

The polymer may be prepared by any method known to those skilled in the art. For example, where the polymer is comprised of a copolymer of lactic and glycolic acid, this copolymer can be prepared by the procedure set forth in U.S. Pat. No. 4,293,539. Also, the polymer can be purchased from commercial suppliers. For example, the cellulose polymers, carboxymethylcellulose salt, hydroxypropylmethylcellulose or methylcellulose are available from commercial suppliers such as Sigma Chemical (St. Louis, Mo.). The polymer is present in the liposphere is the amount of from about 0.1% to about 0.5% weight to volume.

The liposphere also contains at least one compound(s). The compound is present in the liposphere in the amount of from about 2.0% to about 3.0% weight to volume. The compound contained in the liposphere can be a drug such as antianginas, antiarrhythmics, antiasthmatic agents, antibiotics, antidiabetics, antifungals, antihistamines, antihypertensives, antiparasitics, antineoplastics, antitumor drugs, antivirals, cardiac glycosides, herbicides, hormones, immunomodulators, monoclonal antibodies, neurotransitters, nucleic acid proteins, radio contrast agents, radionuclides, sedatives, analgesics, steroids, tranquilizers, vaccines, vasopressors, anesthetics, peptides and the like. Prodrugs that undergo conversion to an active drug upon local interactions with the intracellular medium, cells or tissues can also be used in the lipospheres. The compound used in the liposphere can have a melting point of about 90° C. to about 115° C.

If the compound is a drug, such as an anesthetic, the anesthetic can be a local anesthetic. Examples of local anesthetics include, but are not limited to, bupivacaine, ropivacaine, dibucaine, procaine, chloroprocaine, prilocaine, mepivacaine, etidocaine, tetracaine, lidocaine, and xylocaine, as well as anesthetically active derivatives, analogs and mixtures thereof.

The liposphere(s) contained in the pharmaceutical composition of the present invention can contain varying amounts of glyceride(s), phospholipid(s) (natural or synthetic), polymer(s) and compound(s) depending upon (1) the type of phospholipid used (natural versus synthetic); and (2) the amount of compound(s) to be delivered. For example, if a natural phospholipid is used, when the compound, such as bupivacaine, is present in the amount of from about 2.0% to 3.0% weight to volume, then the amount of glyceride(s) present is in the amount of from about 3.0% to about 9.0% to weight to volume, the amount of natural phospholipid(s) present is in the amount of from about 0.5% to less than about 3.0% weight to volume and the amount of polymer(s) present is in the amount of from about 0.1% to about 0.5% weight to volume. If a synthetic phospholipid is used, when the compound is present in the amount of from about 2.0% to less than about 2.5% weight to volume, then the amount of glyceride(s) present is in the amount of from about 3.0% to about 9.0% to weight to volume, the amount of synthetic phospholipid(s) present is in the amount of from about 0.5% to about 3.0% weight to volume and the amount of polymer(s) present is in the amount of from about 0.1% to about 0.5% weight to volume. Alternatively, if the compound is present in the amount of from about 2.5% to about 3.0% weight to volume, then the amount of glyceride(s) present is in the amount of from about 3.0% to about 9.0% weight to volume, the amount of polymer(s) present is in the amount of from about 0.1% to about 0.5% weight to volume and the synthetic phospholipid(s) is present in the amount of from about 0.75% to about 3.0% weight to volume with the proviso that if the glyceride(s) is present in the amount of about 3.0% by weight to volume then said synthetic phospholipid(s) must be present in the amount of more than about 0.75% weight to volume.

In addition to the glyceride(s), phospholipid(s), polymer(s) and compound(s), the lipospheres can also optionally contain one or more of preservatives, antioxidants, buffers or surfactants. More specifically, the liposphere(s) can contain from about 0.01% to about 2.0% weight to volume of a preservative to prevent microbial growth, such as, but not limited to, methylparaben, propylparaben, or benzyl alcohol. The liposphere(s) can also contain from about 0.0001% to about 1% weight to volume of an antioxidant to prevent oxidation of the formulation. Examples of antioxidants that can be used include, but are not limited to, butylated hydroxyanisole (BHA), butylated hydroxytoluene (BHT), ascorbic acid, and sodium metabisulfite. The liposphere can also contain a buffer to control the pH of the liposphere. Examples of buffers that can be used include, but are not limited to, a phosphate buffer (pH of about 5 to about 8). Suitable preservatives, antioxidants and buffers for use in the lipospheres of the present invention can be purchased from Sigma-Aldrich Corp. (St. Louis, Mo.) and J. T. Baker (Phillipsburg, N.J.).

In comparison to lipospheres known in the art, it has been discovered that the presence of at least one polymer in the lipospheres of the present invention imparts improved stability to the lipospheres. Additionally, lipospheres containing at least one polymer(s) and at least one natural phospholipid(s) exhibit a gellation time of at least about 24 hours at room temperature. Lipospheres containing at least one polymer(s) and at least one synthetic phospholipid(s) can exhibit a gellation time of: (1) at least about fifteen (15) days at room temperature; or (2) thirty (30) days at room temperature.

Methods of Making the Lipospheres

In yet another embodiment, the present invention relates to methods of making the lipospheres used in the pharmaceutical compositions described herein. More specifically, the lipospheres can be prepared by placing the glyceride(s), phospholipid(s) (synthetic or natural), compound, a dispersion medium, and optionally, polymer(s), in a single reaction vessel (such as, but not limited to, a 500-mL glass media jar) and then heating the reaction vessel until all of the solid ingredients have melted to form a mixture. The reaction vessel can be heated to a temperature of from about 50° C. to about 115° C., more specifically, from about 85° C. to about 100° C. The dispersion medium is an aqueous phase that the lipospheres are dispersed in. Examples of a dispersion medium that can be used include water, a buffer such as, but not limited to a phosphate buffer. The amount of a dispersion medium that can be used in the method is from about 85% to about 97%.

After all the ingredients have been melted, the mixture is homogenized until a homogenous fine dispersion is obtained. Once the homogenous fine dispersion is obtained, it is rapidly cooled to a temperature of from about 0° C. to about 10° C. As the dispersion is being cooled, it is also mixed until a suspension of lipospheres having an average particle size of at least about 3 microns and not greater than about 450 microns, is formed. Suitable methods of mixing the dispersion include mechanical shaking or stirring.

As mentioned briefly above, if the liposphere is to include at least one polymer(s), then the polymer can be included with all of the ingredients initially in the reaction vessel. Alternatively, the polymer(s) does not have to be included with the other ingredients at the beginning of the process, but can be added later, specifically, during the cooling process but after the lipospheres have formed.

The lipospheres can be prepared using the above-described methods under aseptic conditions. The lipospheres can then be placed in a container, such as a glass vial, and stored at room temperature or at refrigerated conditions, such as from about 2° C. to about 8° C.

Alternatively, after the lipospheres are obtained using the above-described methods, the lipospheres can be lyophilized using techniques known in the art. Optionally, the lyophilized lipospheres can be terminally sterilized using techniques known in the art, such as, but not limited to, gamma-irradiation and then stored at room temperature or at refrigerated conditions, such as from about 2° C. to about 8° C.

Methods for Inducing or Maintaining Local Anesthesia in a Patient

In yet another embodiment, the present invention relates to methods for inducing or maintaining local anesthesia or sedation in a patient. The method involves administering to a patient an effective amount of a sterile pharmaceutical composition to induce or maintain anesthesia. The pharmaceutical composition administered to the patient comprises at least one liposphere. The liposphere contained in the pharmaceutical composition is one of the previously described lipospheres that contains a compound, such as drug, that is capable of inducing or maintaining anesthesia or sedation. Examples of drugs that induce or maintain anesthesia include, but are not limited to, bupivacaine, ropivacaine, dibucaine, procaine, chloroprocaine, prilocaine, mepivacaine, etidocaine, tetracaine, lidocaine, and xylocaine, as well as anesthetically active derivatives, analogs and mixtures thereof.

The pharmaceutical composition described herein can be administered to a patient topically, enterally (such as orally or rectally), parenterally (subcutaneously, intramuscularly, intraperitoneally) or via local infiltration (i.e. at an open wound site or incision). In addition to the lipospheres, the pharmaceutical composition can also contain a pharmaceutically acceptable carrier or excipient. Suitable excipients include but are not limited to fillers such as sugars, including lactose, sucrose, mannitol, sorbitol, and the like, cellulose preparations such as, maize starch, wheat starch, rice starch, potato starch, gelatin, gum tragacanth, ethyl cellulose, hydroxypropylmethylcellulose, sodium carboxymethylcellulose, polyvinylpyrrolidone (PVP), and the like, as well as mixtures of any two or more. Optionally, disintegrating agents can be included, such as cross-linked polyvinyl pyrrolidone, agar, alginic acid or a salt thereof, such as sodium alginate and the like.

In addition to the excipients, the pharmaceutical composition can include one or more of the following, carrier proteins such as serum albumin, buffers, binding agents, sweeteners and other flavoring agents, preservatives, coloring agents and polyethylene glycol.

As used herein, the term “effective amount” means an amount that produces the effects for which it is administered. The exact dose will be ascertainable by one skilled in the art. As known in the art, adjustments based on age, body weight, sex, diet, time of administration, drug interaction and severity of condition may be necessary and will be ascertainable with routine experimentation by those skilled in the art.

By way of example, and not of limitation, examples of the present invention shall now be given.

### EXAMPLE 1

### Preparation of Liposphere Formulation Containing Bupivacaine (3%) Tristearin (6%), and DMPC (0.75%)

Six grams of bupivacaine free base, 12 grams of tristearin, 1.5 grams of dimyristoylphosphatidylcholine (“DMPC”), and 160 mLs of phosphate buffer (pH 7.4, 50 mM) were added into a 500-mL glass media jar. The mixture was heated to about 90° C. in a water bath until all of the solid components completely melted. The mixture was subsequently transferred into the pre-heated jacketed beaker and homogenized using the high-shear mixer at 8,000 rpm for about five minutes. The hot emulsion was poured into the pre-chilled jacketed beaker set at about 0° C. The dispersion was stirred until its temperature dropped to 10° C., resulting in a formation of lipospheres.

The formulation was examined under an optical microscope and a scanning electron microscope. The particles were spherical in shape with a smooth surface. The particle size distribution of lipospheres was determined by using a Horiba LA920 Laser-Scattering Particle-Size Distribution Analyzer. The diameter ranged from about 1 to about 300 μm

### EXAMPLE 2

### Determination of Gellation Time of Liposphere Formulation Containing Bupivacaine (3%), Tristearin (6%), and DMPC (0.75%)

Approximately 50 mL of the formulation prepared in Example 1 was transferred into a 60 mL glass jar and stored at room temperature. At each sampling time point, the jar was tilted 90°. The formulation was considered non-gelled if it flowed freely without agitation. The formulation was considered gelled if it exhibited resistance to flowing freely when tilted or physically appeared to have solidified, coalesced, coagulated, or phase separated. During the first eight hours of the gellation study, the formulation was tested approximately every hour. After the first day of monitoring the formulation was tested twice daily. Upon a week of monitoring, it was tested approximately every three days and after two weeks, it was tested on a weekly basis. FIG. 1 shows the non-gelled and the gelled samples.

The formulation containing 3% bupivacaine, 6% tristearin, and 0.75% DMPC gelled after 58 days at room temperature.

### EXAMPLE 3

### Gellation Time of Liposphere Formulation Containing Bupivacaine Tristearin, and EPC

Liposphere formulation was made as in Example 1 except that six grams of egg phosphatidylcholine (“EPC”) was used in place of DMPC. The gellation time of this formulation was determined using a procedure described in Example 2. The formulation gelled within two hours at room temperature.

### EXAMPLE 4

### Comparison of Liposphere Formulations with Different Types and Concentrations of Triglyceride and Phospholipid

Liposphere formulations containing bupivacaine free base (3%), and various types of triglyceride (6%) and phospholipid (0.75% or 3%) were prepared as in Example 1. Three types of triglyceride under evaluation were tricaprin (“TC”), trilaurin (“TL”), and tristearin (“TS”). Synthetic phospholipids were DMPC, dipalmitoylphosphatidylcholine (“DPPC”), and distearoylphosphatidylcholine (“DSPC”). Natural phospholipids were EPC and soy phosphatidylcholine (“SPC”). Their gellation times, determined as in Example 2, are given in Table 1, below.

All of the natural-phospholipid-containing formulations gelled within two hours while the formulations with synthetic phospholipid remained liquid for at least about two months at room temperature.

TABLE 1

| | | |
|---|---|---|
| Triglyceride (Conc.) | Phospholipid (Conc.) | Gellation Time |

| | | | | | |
|---|---|---|---|---|---|
| TC | (6%) | DMPC | (0.75%) | >36 | weeks |
| TL | (6%) | DMPC | (0.75%) | >13 | weeks |
| TS | (6%) | DMPC | (0.75%) | 58 | days |
| TS | (6%) | DPPC | (0.75%) | >15 | weeks |
| TS | (6%) | DSPC | (0.75%) | >15 | weeks |
| TS | (6%) | DMPC | (3%) | >15 | weeks |
| TC | (6%) | EPC | (3%) | 2 | hours |
| TL | (6%) | EPC | (3%) | 0.5 | hour |
| TS | (6%) | EPC | (3%) | 2 | hours |
| TS | (6%) | SPC | (3%) | 0.5 | hour |

### EXAMPLE 5

### Comparison of Liposphere Formulations with Different Concentrations of Bupivacaine and Phospholipid

The formulations containing bupivacaine free base (2% or 3%), tristearin (6%), and phospholipid (0.75% to 3.0% of EPC or DMPC) were prepared as in Example 1 and their gellation times were determined as in Example 2. Table 2, below, lists the gellation times of the formulations stored at room temperature. The formulations containing EPC all gelled within 21 hours. The gellation time of the formulation containing 2% bupivacaine, 6% tristearin, and 0.5% EPC was recorded as 0 hour due to the gellation of the sample immediately after the preparation was completed. Some EPC-containing formulations coagulated upon processing. The formulations prepared with DMPC did not gel until at least 58 days. Some DMPC-containing formulations were physically stable for more than 6 months. In general, the formulations with 3% bupivacaine were found to have gelled faster than those containing 2% bupivacaine.

TABLE 2

| | |
|---|---|
| Bupivacaine | Phospholipid (%) |

| | | | |
|---|---|---|---|
| (%) | EPC | DMPC | Gellation Time |

| | | | | |
|---|---|---|---|---|
| 2.0 | 0.5 | — | 0 | hour |
| | — | 0.5 | >134 | days |
| | 0.75 | — | 21 | hours |
| | — | 0.75 | >177 | days |
| | 1.5 | — | 3 | hours |
| | — | 1.5 | >204 | days |
| | 2.0 | — | 3 | hours |
| | — | 2.0 | 123 | days |
| | 3.0 | — | 2 | hours |
| | — | 3.0 | 60 | days |

| | | | |
|---|---|---|---|
| 3.0 | 0.75 | — | Coagulate |

| | | | |
|---|---|---|---|
| — | 0.75 | 58 | days |

| | | |
|---|---|---|
| 1.5 | — | Coagulate |

| | | | |
|---|---|---|---|
| — | 1.5 | 112 | days |
| 2.0 | — | 1 | hour |
| — | 2.0 | 78 | days |
| 3.0 | — | 2 | hours |
| — | 3.0 | 91 | days |

### EXAMPLE 6

### Comparison of Bupivacaine (2%) Liposphere Formulations with Different Concentrations of Tristearin and Phospholipid

The effect of tristearin and phospholipid concentrations on the gelling of liposphere formulations was investigated. The formulations containing bupivacaine (2%) with various amounts of tristearin (3% or 9%) and phospholipid (0.5%, 0.75%, 1.5%, or 3.0%) were prepared as in Example 1 and evaluated as in Example 2. The results are given in Table 3, below.

All the formulations with 3% tristearin and different levels of EPC gelled immediately after they were made. Therefore, their gellation times were recorded as 0 hour. In contrary, the formulations containing DMPC did not gel for at least 4 months. Generally, the DMPC formulations with 9% tristearin were not as physically stable as those prepared with 3% tristearin.

TABLE 3

| | |
|---|---|
| Tristearin | Phospholipid (%) |

| | | | |
|---|---|---|---|
| (%) | EPC | DMPC | Gellation Time |

| | | | | |
|---|---|---|---|---|
| 3.0 | 0.5 | — | 0 | hour |
| | — | 0.5 | 107 | days |
| | 0.75 | — | 0 | hour |
| | — | 0.75 | 149 | days |
| | 1.5 | — | 0 | hour |
| | — | 1.5 | 88 | days |
| | 3.0 | — | 0 | hour |
| | — | 3.0 | 88 | days |

| | | | |
|---|---|---|---|
| 9.0 | 0.5 | — | Phase separation |

| | | | |
|---|---|---|---|
| — | 0.5 | >134 | days |
| 0.75 | — | 6 | hours |
| — | 0.75 | 4 | days |
| 1.5 | — | 0 | hour |
| — | 1.5 | 4 | days |
| 3.0 | — | 0 | hour |
| — | 3.0 | 4 | days |

### EXAMPLE 7

### Comparison of Bupivacaine (3%) Liposphere Formulations with Different Concentrations of Tristearin and Phospholipid

In this example, the effect of tristearin and phospholipid concentrations on the gelling of liposphere formulations containing 3% bupivacaine was investigated. Liposphere formulations with various amounts of tristearin (3% or 9%) and phospholipid (0.75%, 1.5%, or 3.0%) were prepared as in Example 1. The gellation times, determined as in Example 2, are given in Table 4, below.

All of the EPC-containing formulations were unstable. Most of the DMPC-containing formulations remained liquid for more than 24 hours. However, the formulation with 3% tristearin and 0.75% DMPC coagulated and that with 9% tristearin and 3% DMPC gelled after six hours.

TABLE 4

| | | |
|---|---|---|
| Tristearin | Phospholipid (%) | Gellation Time |

| | | | |
|---|---|---|---|
| (%) | EPC | DMPC | Room Temperature |

| | | | | |
|---|---|---|---|---|
| 3.0 | 0.75 | — | 0 | hour |

| | | |
|---|---|---|
| — | 0.75 | Coagulate |

| | | | | |
|---|---|---|---|---|
| | 1.5 | — | 0 | hour |
| | — | 1.5 | 149 | days |
| | 3.0 | — | 0 | hour |
| | — | 3.0 | 88 | days |
| 9.0 | 0.75 | — | 0 | hour |
| | — | 0.75 | 11 | days |
| | 1.5 | — | 0 | hour |
| | — | 1.5 | 11 | days |
| | 3.0 | — | 0 | hour |
| | — | 3.0 | 6 | hours |

### EXAMPLE 8

### Comparison of Bupivacaine (2.5%) Liposphere Formulations with Different Concentrations of Tristearin and Phospholipid

Liposphere formulations containing bupivacaine free base (2.5%) with tristearin (3% or 9%), and phospholipid (0.75%, 1.5%, or 3.0%) were prepared as in Example 1 and evaluated for their gellation times as in Example 2.

The gellation behavior of these 2.5% bupivacaine liposphere formulations (Table 5, below) was similar to that of the formulation containing 3% bupivacaine. All of the EPC-containing formulations gelled before the gellation study was initiated. The DMPC-containing formulations remained liquid for at least four days, with an exception of the formulation with 3% tristearin and 0.75% DMPC, which coagulated.

TABLE 5

| | | |
|---|---|---|
| Tristearin | Phospholipid (%) | Gellation Time |

| | | | |
|---|---|---|---|
| (%) | EPC | DMPC | Room Temperature |

| | | | | |
|---|---|---|---|---|
| 3.0 | 0.75 | — | 0 | hour |

| | | |
|---|---|---|
| — | 0.75 | Coagulate |

| | | | | |
|---|---|---|---|---|
| | 1.5 | — | 0 | hour |
| | — | 1.5 | >126 | days |
| | 3.0 | — | 0 | hour |
| | — | 3.0 | >126 | days |
| 9.0 | 0.75 | — | 0 | hour |
| | — | 0.75 | 9 | days |
| | 1.5 | — | 0 | hour |
| | — | 1.5 | 5 | days |
| | 3.0 | — | 0 | hour |
| | — | 3.0 | 4 | days |

### EXAMPLE 9

### Preparation of Liposphere Formulations with Addition of Carboxymethylcellulose

Six grams of bupivacaine free base, 12 grams of tristearin, 1.5 grams of DMPC, and 140 mLs of phosphate buffer (pH 7.4, 50 mM) were added into a 500 mL glass media jar. The mixture was heated to about 90° C. in a water bath until all of the solid components completely melted. The mixture was subsequently transferred into the preheated jacketed beaker and homogenized using the high-shear mixer at about 8,000 rpm for about five minutes. The emulsion was poured into the pre-chilled jacketed beaker set at about 0° C. The dispersion was stirred until its temperature dropped to 10° C., resulting in a formation of lipospheres. Subsequently, 20 mLs of 5% Carboxymethylcellulose (“CMC”) in phosphate buffer solution was gradually stirred into the liposphere formulation. The final concentration of CMC in this formulation was 0.5%.

The resulted formulation was a homogeneous dispersion of solid liposphere particles. The gellation time of this formulation determined according to Example 2 was greater than 84 days.

### EXAMPLE 10

### Comparison of Bupivacaine (2%) Liposphere Formulations with CMC and Different Concentrations of Tristearin and DMPC

Liposphere formulations containing bupivacaine free base (2%) with CMC (0.5%), tristearin (3%, 6% or 9%), and DMPC (0.5%, 0.75%, or 3.0%) were prepared as in Example 9 and their gellation times were determined as in Example 2. All of the formulations did not gel for at least three weeks. The results are shown below in Table 6.

TABLE 6

| | | |
|---|---|---|
| Tristearin (%) | DMPC (%) | Gellation Time |

| | | | |
|---|---|---|---|
| 3.0 | 0.5 | >72 | days |
| | 3.0 | >72 | days |
| 6.0 | 0.75 | >84 | days |
| | 3.0 | >84 | days |
| 9.0 | 0.75 | >72 | days |
| | 3.0 | 21 | days |

### EXAMPLE 11

### Comparison of Bupivacaine (3%) Liposphere Formulations with CMC and Different Concentrations of Tristearin and DMPC

Liposphere formulations containing bupivacaine free base (3%) with CMC (0.5%), tristearin (3%, 6% or 9%), and DMPC (0.75%, or 3.0%) were prepared as in Example 9. The gellation times, determined as in Example 2, are given in Table 7 below. All formulations remained liquid dispersions for at least 72 days except the formulation containing 3% tristearin and 0.75% DMPC which coagulated.

TABLE 7

| Tristearin (%) | DMPC (%) | Gellation Time |
|---|---|---|
| 3.0 | 0.75 | coagulate |
| | 3.0 | >72 days |
| 6.0 | 0.75 | >84 days |
| | 3.0 | >84 days |
| 9.0 | 0.75 | >72 days |
| | 3.0 | >72 days |

### EXAMPLE 12

### Comparison of Bupivacaine Liposphere Formulations with CMC and Difference Concentrations of Tristearin and EPC

Liposphere formulations containing bupivacaine free base (2% or 3%) with CMC (0.5%), tristearin (3%, 6% or 9%), and EPC (0.5% or 0.75%) were prepared as in Example 9. The gellation times, determined as in Example 2, are given in Table 8, below. All formulations remained liquid dispersions for at least 24 hours.

TABLE 8

| | | | |
|---|---|---|---|
| Bupivacaine | Tristearin | EPC | |
| (%) | (%) | (%) | Gellation Time |

| | | | | |
|---|---|---|---|---|
| 2 | 3.0 | 0.5 | >72 | days |
| 2 | 9.0 | 0.75 | 24 | hours |
| 3 | 6.0 | 0.75 | >84 | days |

### EXAMPLE 13

### Preparation of Liposphere Formulations with Addition of Carboxymethylcellulose (CMC) prior to Liposphere Formation

Six grams of bupivacaine free base, 12 grams of tristearin, 1.5 grams of DMPC, 20 mLs of 5% CMC in phosphate buffer solution, and 140 mLs of phosphate buffer (pH 7.4, 50 mM) were added into a 500 mL glass media jar. The mixture was heated to about 90° C. in a water bath until all of the solid components completely melted. The mixture was subsequently transferred into the pre-heated jacketed beaker and homogenized using the high-shear mixer at about 8,000 rpm for about five minutes. The emulsion was poured into the pre-chilled jacketed beaker set at about 0° C. The dispersion was stirred until its temperature dropped to about 10° C., resulting in a formation of lipospheres.

The resulted formulation was a homogeneous dispersion of solid liposphere particles. The gellation time of this formulation determined according to Example 2 was greater than 43 days.

### EXAMPLE 14

### Comparison of Bupivacaine (2%) Liposphere Formulations with CMC and Different Concentrations of Tristearin and DMPC

Liposphere formulations containing bupivacaine free base (2%) with CMC (0.5%), tristearin (3%, 6% or 9%), and DMPC (0.5%, 0.75%, or 3.0%) were prepared as in Example 13 and their gellation times were determined as in Example 2. All of the formulations did not gel for at least four weeks. The results are shown below in Table 9.

TABLE 9

| Tristearin (%) | DMPC (%) | Gellation Time |
|---|---|---|
| 3.0 | 0.5 | >65 days |
| | 3.0 | >65 days |
| 6.0 | 0.75 | >70 days |
| | 3.0 | >70 days |
| 9.0 | 0.75 | >65 days |
| | 3.0 | 28 days |

### EXAMPLE 15

### Comparison of Bupivacaine (3%) Liposphere Formulations with CMC and Different Concentrations of Tristearin and DMPC

Liposphere formulations containing bupivacaine free base (3%) with CMC (0.5%), tristearin (3%, 6% or 9%), and DMPC (0.75%, or 3.0%) were prepared as in Example 13. The gellation times, determined as in Example 2, are given in Table 10, below. All formulations remained liquid dispersions for at least 38 days.

TABLE 10

| Tristearin (%) | DMPC (%) | Gellation Time |
|---|---|---|
| 3.0 | 0.75 | >65 days |
| | 3.0 | >65 days |
| 6.0 | 0.75 | >70 days |
| | 3.0 | >70 days |
| 9.0 | 0.75 | >15 days |
| | 3.0 | 38 days |

### EXAMPLE 16

### Rat Thermal Paw Flick Anesthesia Study of Bupivacaine Formulations

Liposphere containing bupivacaine (3%), tristearin (6%), and DMPC (0.75%) was tested for its local anesthetic activity in rats. Its activity was compared with that of bupivacaine HCL (3%) solution. The liposphere formulation was prepared as described in Example 1.

Adult rats were given an intraplantar injection of 100 μL of either liposphere formulation or bupivacaine HCl solution. Animals were then individually placed into a Hargreaves “hotbox,” in which a beam of light, as a heat source, was focused on the plantar surface of the injected paw at various intervals after treatment. The latency for the rat to withdraw its paw was recorded, up to a maximum of 20 seconds. The earliest time point examined in these studies was 30 min. The latest time point was 120 min for animals treated with bupivacaine HCl solution (by which time latencies had returned to predrug baseline levels), and 1440 min for animals treated with liposphere formulation.

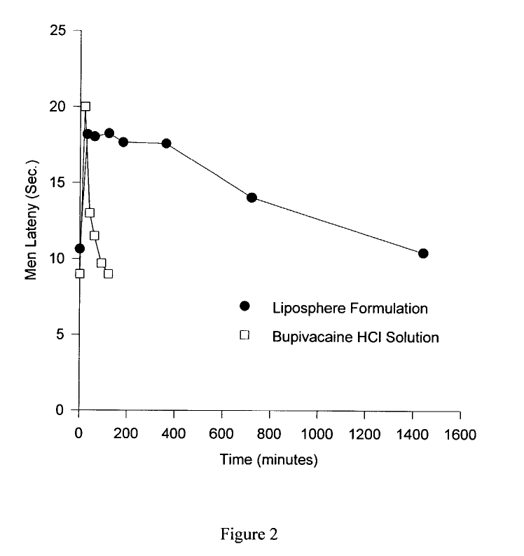

The results of these studies are indicated in FIG. 2. Rats given liposphere formulation demonstrated anesthesia for up to 720 min post injection. Rats given bupivacaine HCl solution demonstrated the presence of anesthesia/analgesia for up to 60 minutes post injection.

### EXAMPLE 17

### Guinea Pig Wheal Anesthesia Study of Bupivacaine Formulations

Liposphere formulation containing bupivacaine (3%), tristearin (6%), and DMPC (0.75%) and the liposphere vehicle containing tristearin (6%) and DMPC (0.75%) were evaluated for their anesthesia activities in guinea pigs. The liposphere formulation and vehicle were prepared as described in Example 1. In addition, a solution of bupivacaine HCl (0.5%) and its vehicle (normal saline solution) were evaluated for comparison.

In this example, adult guinea pigs were randomly assigned to one of two groups, with each group receiving two subcutaneous injections: one of vehicle and one of the test articles. Half of the animals in each group were given the vehicle on the left side and the test article on the right side. The other half were given the test article on the left side and the vehicle on the right. Injection volume was 300 μL. The treatment groups were as indicated in Table 11, below.

TABLE 11

| Group (N) | Injection 1 | Injection 2 | Dose Volume |
|---|---|---|---|
| 1 (8) | Bupivacaine (3%) | Liposphere vehicle | 0.3 mL |
| | liposphere formulation | | |
| 2 (8) | Bupivacaine HCl | Normal saline | 0.3 mL |
| | (0.5%) solution | | |

There were two injections given to each animal. Both groups consisted of eight animals, four of each group received an injection of the vehicle on the left side of the spine, and the remaining four animals received an injection of the vehicle on the right side. Injection of test article was made on the side opposite to the vehicle injection site. An ink marker was used to mark the injection sites of approximately 10 mm in diameter.

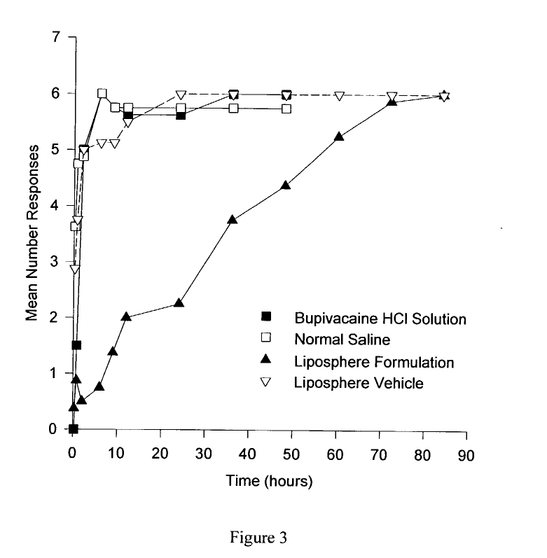

The “anesthetic action” (the ability of local anesthetics to block the skin reflex in guinea pigs) was determined using the “pin-prick” method. This method is based on the observation that mechanical probing of the skin on the back of guinea pigs elicits a contraction (twitch response) of the skin at the site of probing. The ability of the test compound to block the twitch responses of the guinea pig to six probings with a carefully calibrated needle algesimeter was evaluated. The average number of probings not producing a dermal twitch response was designated as the anesthesia score. The algesimeter was calibrated at 20 grams, with the use of a scale, in these experiments. The algesimeter was used to assess onset and depth of anesthesia 0.25, 0.75, 2, 6, 9, 12, 24, 36, 48, 60, 72 and 84 hours after the subcutaneous injection of each solution. The results are shown in FIG. 3.

The bupivacaine (3%) liposphere formulation promoted statistically significant anesthesia compared to its vehicle up to 48 hours [p<0.043; 1-tailed test]. Treatment with bupivacaine HCl solution promoted statistically significant anesthesia compared to its vehicle up to 0.75 hour post dosing [p<0.008; 1-tailed test].

All abstracts, references, patents and published patent applications referred to herein are hereby incorporated by reference.

The present invention is illustrated by way of the foregoing description and examples. The foregoing description is intended as a non-limiting illustration, since many variations will become apparent to those skilled in the art in view thereof.

Changes can be made to the composition, operation and arrangement of the method of the present invention described herein without departing from the concept and scope of the invention.

What is claimed is:

1. A pharmaceutical composition comprising:
lipospheres, wherein each of said lipospheres comprises at least one glyceride and at least one synthetic phospholipid, said glyceride being present in an amount of from about 3.0% to about 9.0% weight to volume, and said synthetic phospholipid being present in an amount of from about 0.5% to about 3.0% weight to volume;
wherein said lipospheres have an average particle size of at least about 3 microns and not greater than about 450 microns;
wherein at least one of said lipospheres comprises a compound, said compound being present in an amount of from about 2.0% to less than about 2.5% weight to volume; and
wherein at least one of the lipospheres has a gel time of at least 24 hours at room temperature.

2. The pharmaceutical composition of claim 1 wherein the compound is a drug.

3. The pharmaceutical composition of claim 2 wherein the drug contains bupivacaine.

4. The pharmaceutical composition of claim 1 wherein the glyceride is a monoglyceride, a diglyceride or a triglyceride.

5. The pharmaceutical composition of claim 4 wherein the glyceride is a triglyceride.

6. The pharmaceutical composition of claim 5 wherein the triglyceride is tristearin, trilaurin, tricaprin, triarachidin or trimyristin.

7. The pharmaceutical composition of claim 1 wherein the synthetic phospholipid is dimyristoylphosphatidylcholine, dipalmitoylphosphatidylcholine, distearoylphosphatidylcholine, dilauroylphosphatidylcholine, or diarachidoylphosphatidylcholine.

8. A pharmaceutical composition comprising:
lipospheres, wherein each of said lipospheres comprises at least one glyceride and at least one synthetic phospholipid, said glyceride being present in an amount of from about 3.0% to about 9.0% weight to volume, and said synthetic phospholipid being present in an amount of from about 0.75% to about 3.0% weight to volume with the proviso that if the glyceride is present in the amount of 3.0% weight to volume then said synthetic phospholipid must be present in the amount of more than about 0.75% by weight to volume and if the glyceride is present in the amount of about 9.0% by weight to volume then said synthetic phospholipid must be present in the amount of less than about 3.0% weight to volume;
wherein said lipospheres have an average particle size of at least about 3 microns and not greater than about 450 microns;
wherein in at least one of said lipospheres comprises a compound, said compound being present in an amount of from about 2.5% to about 3.0% weight to volume; and
wherein at least one of the lipospheres has a gel time of at least 24 hours at room temperature.

9. The pharmaceutical composition of claim 8 wherein the compound is a drug.

10. The pharmaceutical composition of claim 9 wherein the drug contains bupivacaine.

11. The pharmaceutical composition of claim 8 wherein the glyceride is a monoglyceride, a diglyceride or a triglyceride.

12. The pharmaceutical composition of claim 11 wherein the glyceride is a triglyceride.

13. The pharmaceutical composition of claim 12 wherein the triglyceride is tristearin, trilaurin, tricaprin, triarachidin or trimyristin.

14. The pharmaceutical composition of claims 8 wherein the synthetic phospholipid is dimyristoylphosphatidylcholine, dipalmitoylphosphatidylcholine, distearoylphosphatidylcholine, dilauroylphosphatidylcholine, or diarachidoylphosphatidylcholine.

15. A pharmaceutical composition comprising:
lipospheres, wherein each of said lipospheres comprises at least one glyceride, at least one natural phospholipid and a polymer, said glyceride being present in an amount of from about 3.0% to about 9.0% weight to volume, said natural phospholipid being present in an amount of from about 0.5% to less than about 3.0% weight to volume, and said polymer being present in an amount of from about 0.1% to about 0.5% weight to volume;
wherein said lipospheres have an average particle size of at least about 3 microns and not greater than about 450 microns;
wherein at least one of said lipospheres comprises a compound, said compound being present in an amount of from about 2.0% to about 3.0% weight to volume; and
wherein at least one of the lipospheres has a gel time of at least 24 hours at room temperature.

16. The pharmaceutical composition of claim 15 wherein the polymer is gelatin, polyvinyl pyrrolidone, polyethylene glycol, alginate or a cellulose polymer.

17. The pharmaceutical composition, of claim 16 wherein the cellulose polymer is carboxymethylcellulose salt, hydroxypropylmethylcellulose or methylcellulose.

18. The pharmaceutical composition of claim 15 wherein the compound is a drug.

19. The pharmaceutical composition of claim 18 wherein the drug contains bupivacaine.

20. The pharmaceutical composition of claim 15 wherein the glyceride is a monoglyceride, a diglyceride or a triglyceride.

21. The pharmaceutical composition of claim 20 wherein the glyceride is a triglyceride.

22. The pharmaceutical composition of claim 21 wherein the triglyceride is tristearin, trilaurin, tricaprin, triarachidin or trimyristin.

23. The pharmaceutical composition of claim 15 wherein the natural phospholipid is egg phosphatidylcholine or soy phosphatidylcholine.

24. A pharmaceutical composition comprising:
lipospheres, wherein each of said lipospheres comprises at least one glyceride, at least one synthetic phospholipid and a polymer, said glyceride being present in an amount of from about 3.0% to about 9.0% weight to volume, said synthetic phospholipid being present in an amount of from about 0.5% to about 3.0% weight to volume, and said polymer being present in an amount of from about 0.1% to about 0.5% weight to volume;
wherein said lipospheres have an average particle size of at least about 3 microns and not greater than about 450 microns;
wherein at least one of said lipospheres comprises a compound, said compound being present in an amount of from about 2.0% to less than about 2.5% weight to volume; and
wherein at least one of the lipospheres has a gel time of at least 15 days at room temperature.

25. The pharmaceutical composition of claim 24 wherein the polymer is gelatin, polyvinyl pyrrolidone, polyethylene glycol, alginate or a cellulose polymer.

26. The pharmaceutical composition of claim 25 wherein the cellulose polymer is carboxymethylcellulose salt, hydroxypropylmethylcellulose or methylcellulose.

27. The pharmaceutical composition of claim 24 wherein the compound is a drug.

28. The pharmaceutical composition of claim 27 wherein the drug contains bupivacaine.

29. The pharmaceutical composition of claim 24 wherein the glyceride is a monoglyceride, a diglyceride or a triglyceride.

30. The pharmaceutical composition of claim 29 wherein the glyceride is a triglyceride.

31. The pharmaceutical composition of claim 30 wherein the triglyceride is tristearin, trilaurin, tricaprin, triarachidin or trimyristin.

32. The pharmaceutical composition of claim 24 wherein the synthetic phospholipid is dimyristoylphosphatidylcholine, dipalmitoylphosphatidylcholine, distearoylphosphatidylcholine, dilauroylphosphatidylcholine, or diarachidoylphosphatidylcholine.

33. A pharmaceutical composition comprising:
lipospheres, wherein each of said lipospheres comprises at least one glyceride, at least one synthetic phospholipid and a polymer, said glyceride being present in an amount of from about 3.0% to about 9.0% weight to volume, said synthetic phospholipid being present in an amount of from about 0.75% to about 3.0% weight to volume with the proviso that if the glyceride is present in the amount of about 3.0% by weight to volume then said synthetic phospholipid(s) must be present in the amount of more than about 0.75% weight to volume and said polymer being present in the amount of from about 0.1% to about 0.5% weight to volume;
wherein said lipospheres have an average particle size of at least about 3 microns and not greater than about 450 microns;
wherein in at least one of said lipospheres comprises a compound, said compound being present in an amount of from about 2.5% to about 3.0% weight to volume; and
wherein at least one of the lipospheres has a gel time of at least 30 days at room temperature.

34. The pharmaceutical composition of claim 33 wherein the polymer is gelatin, polyvinyl pyrrolidone, polyethylene glycol, alginate or a cellulose polymer.

35. The pharmaceutical composition of claim 34 wherein the cellulose polymer is carboxymethylcellulose salt, hydroxypropylmethylcellulose or methylcellulose.

36. The pharmaceutical composition of claim 33 wherein the compound is a drug.

37. The pharmaceutical composition of claim 36 wherein the drug contains bupivacaine.

38. The pharmaceutical composition of claim 33 wherein the glyceride is a monoglyceride, a diglyceride or a triglyceride.

39. The pharmaceutical composition of claim 38 wherein the glyceride is a triglyceride.

40. The pharmaceutical composition of claim 39 wherein the triglyceride is tristearin, trilaurin, tricaprin, triarachidin or trimyristin.

41. The pharmaceutical composition of claims 33 wherein the synthetic phospholipid is dimyristoylphosphatidylcholine, dipalmitoylphosphatidylcholine, distearoylphosphatidylcholine, dilauroylphosphatidylcholine, or diarachidoylphosphatidylcholine.

42. A pharmaceutical composition comprising:
lipospheres, wherein each of said lipospheres comprises at least one triglyceride and at least one synthetic phospholipid, said triglyceride being present in an amount of from about 3.0% to about 9.0% weight to volume, and said synthetic phospholipid being present in an amount of from about 0.5% to about 3.0% weight to volume;
wherein said lipospheres have an average particle size of at least about 3 microns and not greater than about 450 microns;
wherein at least one of said lipospheres comprises a drug, said drug being present in an amount of from about 2.0% to less than about 2.5% weight to volume; and
wherein at least one of the lipospheres has a gel time of at least 24 hours at room temperature.

43. The pharmaceutical composition of claim 42 wherein the drug contains bupivacaine.

44. The pharmaceutical composition of claim 42 wherein the triglyceride is tristearin, trilaurin, tricaprin, triarachidin or trimyristin.

45. The pharmaceutical composition of claim 44 wherein the synthetic phospholipid is dimyristoylphosphatidylcholine, dipalmitoylphosphatidylcholine, distearoylphosphatidylcholine, dilauroylphosphatidylcholine, or diarachidoylphosphatidylcholine.

46. A pharmaceutical composition comprising:
lipospheres, wherein each of said lipospheres comprises at least one triglyceride and at least one synthetic phospholipid, said triglyceride being present in an amount of from about 3.0% to about 9.0% weight to volume, and said synthetic phospholipid being present in an amount of from about 0.75% to about 3.0% weight to volume with the proviso that if the triglyceride is present in the amount of 3.0% weight to volume then said synthetic phospholipid must be present in the amount of more than about 0.75% by weight to volume and if the triglyceride is present in the amount of about 9.0% by weight to volume then said synthetic phospholipid must be present in the amount of less than about 3.0% weight to volume;
wherein said lipospheres have an average particle size of at least about 3 microns and not greater than about 450 microns;
wherein in at least one of said lipospheres comprises a drug, said drug being present in an amount of from about 2.5% to about 3.0% weight to volume; and
wherein at least one of the lipospheres has a gel time of at least 24 hours at room temperature.

47. The pharmaceutical composition of claim 46 wherein the drug contains bupivacaine.

48. The pharmaceutical composition of claim 46 wherein the triglyceride is tristearin, trilaurin, tricaprin, triarachidin or trimyristin.

49. The pharmaceutical composition of claims 46 wherein the synthetic phospholipid is dimyristoylphosphatidylcholine, dipalmitoylphosphatidylcholine, distearoylphosphatidylcholine, dilauroylphosphatidylcholine, or diarachidoylphosphatidylcholine.

50. A pharmaceutical composition comprising:
lipospheres, wherein each of said lipospheres comprises at least one triglyceride, at least one natural phospholipid and a polymer, said triglyceride being present in an amount of from about 3.0% to about 9.0% weight to volume, said natural phospholipid being present in an amount of from about 0.5% to less than about 3.0% weight to volume, and said polymer being present in an amount of from about 0.1% to about 0.5% weight to volume;
wherein said lipospheres have an average particle size of at least about 3 microns and not greater than about 450 microns;
wherein at least one of said lipospheres comprises a drug, said compound being present in an amount of from about 2.0% to about 3.0% weight to volume; and
wherein at least one of the lipospheres has a gel time of at least 24 hours at room temperature.

51. The pharmaceutical composition of claim 50 wherein the polymer is gelatin, polyvinyl pyrrolidone, polyethylene glycol, alginate or a cellulose polymer.

52. The pharmaceutical composition of claim 51 wherein the cellulose polymer is carboxymethylcellulose salt, hydroxypropylmethylcellulose or methylcellulose.

53. The pharmaceutical composition of claim 50 wherein the drug contains bupivacaine.

54. The pharmaceutical composition of claim 50 wherein the triglyceride is tristearin, trilaurin, tricaprin, triarachidin or trimyristin.

55. The pharmaceutical composition of claim 50 wherein the natural phospholipid is egg phosphatidylcholine or soy phosphatidylcholine.

56. A pharmaceutical composition comprising:
lipospheres, wherein each of said lipospheres comprises at least one triglyceride, at least one synthetic phospholipid and a polymer, said triglyceride being present in an amount of from about 3.0% to about 9.0% weight to volume, said synthetic phospholipid being present in an amount of from about 0.5% to about 3.0% weight to volume, and said polymer being present in an amount of from about 0.1% to about 0.5% weight to volume;
wherein said lipospheres have an average particle size of at least about 3 microns and not greater than about 450 microns;
wherein at least one of said lipospheres comprises a drug, said drug being present in an amount of from about 2.0% to less than about 2.5% weight to volume; and
wherein at least one of the lipospheres has a gel time of at least 15 days at room temperature.

57. The pharmaceutical composition of claim 56 wherein the polymer gelatin, polyvinyl pyrrolidone, polyethylene glycol, alginate or a cellulose polymer.

58. The pharmaceutical composition of claim 57 wherein the cellulose polymer is carboxymethylcellulose salt, hydroxypropylmethylcellulose or methylcellulose.

59. The pharmaceutical composition of claim 56 wherein the drug contains bupivacaine.

60. The pharmaceutical composition of claim 56 wherein the triglyceride is tristearin, trilaurin, tricaprin, triarachidin or trimyristin.

61. The pharmaceutical composition of claim 56 wherein the synthetic phospholipid is dimyristoylphosphatidylcholine, dipalmitoylphosphatidylcholine, distearoylphosphatidylcholine, dilauroylphosphatidylcholine, or diarachidoylphosphatidylcholine.

62. A pharmaceutical composition comprising:
lipospheres, wherein each of said lipospheres comprises at least one triglyceride, at least one synthetic phospholipid and a polymer, said triglyceride being present in an amount of from about 3.0% to about 9.0% weight to volume, said synthetic phospholipid being present in an amount of from about 0.75% to about 3.0% weight to volume with the proviso that if the glyceride is present in the amount of about 3.0% by weight to volume then said synthetic phospholipid(s) must be present in the amount of more than about 0.75% weight to volume and said polymer being present in the amount of from about 0.1% to about 0.5% weight to volume;
wherein said lipospheres have an average particle size of at least about 3 microns and not greater than about 450 microns;
wherein in at least one of said lipospheres comprises a compound, said compound being present in an amount of from about 2.5% to about 3.0% weight to volume; and
wherein at least one of the lipospheres has a gel time of at least 30 days at room temperature.

63. The pharmaceutical composition of claim 62 wherein the polymer is gelatin, polyvinyl pyrrolidone, polyethylene glycol, alginate or a cellulose polymer.

64. The pharmaceutical composition of claim 63 wherein the cellulose polymer is carboxymethylcellulose salt, hydroxypropylmethylcellulose or methylcellulose.

65. The pharmaceutical composition of claim 62 wherein the drug contains bupivacaine.

66. The pharmaceutical composition of claim 62 wherein the triglyceride is tristearin, trilaurin, tricaprin, triarachidin or trimyristin.

67. The pharmaceutical composition of claims 62 wherein the synthetic phospholipid is dimyristoylphosphatidylcholine, dipalmitoylphosphatidylcholine, distearoylphosphatidylcholine, dilauroylphosphatidylcholine, or diarachidoylphosphatidylcholine.

68. A pharmaceutical composition comprising:
lipospheres, wherein each of said lipospheres comprises at least one triglyceride and at least one synthetic phospholipid, said triglyceride being present in an amount of from about 3.0% to about 9.0% weight to volume, and said synthetic phospholipid being present in an amount of from about 0.5% to about 3.0% weight to volume;
wherein said lipospheres have an average particle size of at least about 3 microns and not greater than about 450 microns;
wherein at least one of said lipospheres comprises a drug containing bupivacaine, said drug being present in the amount of from about 2.0% to less than about 2.5% weight to volume; and
wherein at least one of the lipospheres has a gel time of at least 24 hours at room temperature.

69. The pharmaceutical composition of claim 68 wherein the triglyceride is tristearin, trilaurin, tricaprin, triarachidin or trimyristin.

70. The pharmaceutical composition of claim 68 wherein the synthetic phospholipid is dimyristoylphosphatidylcholine, dipalmitoylphosphatidylcholine, distearoylphosphatidylcholine, dilauroylphosphatidylcholine, or diarachidoylphosphatidylcholine.

71. A pharmaceutical composition comprising:
lipospheres, wherein each of said lipospheres comprises at least one triglyceride and at least one synthetic phospholipid, said triglyceride being present in an amount of from about 3.0% to about 9.0% weight to volume, and said synthetic phospholipid being present in an amount of from about 0.75% to about 3.0% weight to volume with the proviso that if the triglyceride is present in the amount of 3.0% weight to volume then said synthetic phospholipid must be present in the amount of more than about 0.75% by weight to volume and if the triglyceride is present in the amount of about 9.0% by weight to volume then said synthetic phospholipid must be present in the amount of less than about 3.0% weight to volume;
wherein said lipospheres have an average particle size of at least about 3 microns and not greater than about 450 microns;
wherein in at least one of said lipospheres comprises a drug containing bupivacaine, said drug being present in the amount of from about 2.5% to about 3.0% weight to volume; and
wherein at least one of the lipospheres has a gel time of at least 24 hours at room temperature.

72. The pharmaceutical composition of claim 71 wherein the triglyceride is tristearin, trilaurin, tricaprin, triarachidin or trimyristin.

73. The pharmaceutical composition of claims 71 wherein the synthetic phospholipid is dimyristoylphosphatidylcholine, dipalmitoylphosphatidylcholine, distearoylphosphatidylcholine, dilauroylphosphatidylcholine, or diarachidoylphosphatidylcholine.

74. A pharmaceutical composition comprising:
lipospheres, wherein each of said lipospheres comprises at least one triglyceride, at least one natural phospholipid and a polymer, said triglyceride being present in an amount of from about 3.0% to about 9.0% weight to volume, said natural phospholipid being present in an amount of from about 0.5% to less than about 3.0% weight to volume, and said polymer being present in an amount of from about 0.1% to about 0.5% weight to volume;
wherein said lipospheres have an average particle size of at least about 3 microns and not greater than about 450 microns;
wherein at least one of said lipospheres comprises a drug containing bupivacaine, said drug being present in an amount of from about 2.0% to less than about 3.0% weight to volume; and
wherein at least one of the lipospheres has a gel time of at least 24 hours at room temperature.

75. The pharmaceutical composition of claim 74 wherein the polymer is gelatin, polyvinyl pyrrolidone, polyethylene glycol, alginate or a cellulose polymer.

76. The pharmaceutical composition of claim 75 wherein the cellulose polymer is carboxymethylcellulose salt, hydroxypropylmethylcellulose or methylcellulose.

77. The pharmaceutical composition of claim 74 wherein the triglyceride is tristearin, trilaurin, tricaprin, triarachidin or trimyristin.

78. The pharmaceutical composition of claim 74 wherein the natural phospholipid is egg phosphatidylcholine or soy phosphatidylcholine.

79. A pharmaceutical composition comprising:
lipospheres, wherein each of said lipospheres comprises at least one triglyceride, at least one synthetic phospholipid and a polymer, said triglyceride being present in an amount of from about 3.0% to about 9.0% weight to volume, said synthetic phospholipid being present in an amount of from about 0.5% to about 3.0% weight to volume, and said polymer being present in an amount of from about 0.1% to about 0.5% weight to volume;
wherein said lipospheres have an average particle size of at least about 3 microns and not greater than about 450 microns;
wherein at least one of said lipospheres comprises a drug containing bupivacaine, said drug being present in the amount of from about 2.0% to less than about 2.5% weight to volume; and
wherein at least one of the lipospheres has a gel time of at least 15 days at room temperature.

80. The pharmaceutical composition of claim 79 wherein the polymer is gelatin, polyvinyl pyrrolidone, polyethylene glycol, alginate or a cellulose polymer.

81. The pharmaceutical composition of claim 80 wherein the cellulose polymer is carboxymethylcellulose salt, hydroxypropylmethylcellulose or methylcellulose.

82. The pharmaceutical composition of claim 79 wherein the triglyceride is tristearin, trilaurin, tricaprin, triarachidin or trimyristin.

83. The pharmaceutical composition of claim 79 wherein the synthetic phospholipid is dimyristoylphosphatidylcholine, dipalmitoylphosphatidylcholine, distearoylphosphatidylcholine, dilauroylphosphatidylcholine, or diarachidoylphosphatidylcholine.

84. A pharmaceutical composition comprising:
lipospheres, wherein each of said lipospheres comprises at least one triglyceride, at least one synthetic phospholipid and a polymer, said triglyceride being present in an amount of from about 3.0% to about 9.0% weight to volume, said synthetic phospholipid being present in an amount of from about 0.75% to about 3.0% weight to volume with the proviso that if the glyceride is present in the amount of about 3.0% by weight to volume then said synthetic phospholipid(s) must be present in the amount of more than about 0.75% weight to volume and said polymer being present in the amount of from about 0.1% to about 0.5% weight to volume;
wherein said lipospheres have an average particle size of at least about 3 microns and not greater than about 450 microns;
wherein in at least one of said lipospheres comprises a drug containing bupivacaine, said drug being present in the amount of from about 2.5% to about 3.0% weight to volume; and
wherein at least one of the lipospheres has a gel time of at least 30 days at room temperature.

85. The pharmaceutical composition of claim 84 wherein the polymer is gelatin, polyvinyl pyrrolidone, polyethylene glycol, alginate or a cellulose polymer.

86. The pharmaceutical composition of claim 85 wherein the cellulose polymer is carboxymethylcellulose salt, hydroxypropylmethylcellulose or methylcellulose.

87. The pharmaceutical composition of claim 84 wherein the triglyceride is tristearin, trilaurin, tricaprin, triarachidin or trimyristin.

88. The pharmaceutical composition of claims 84 wherein the synthetic phospholipid is dimyristoylphosphatidylcholine, dipalmitoylphosphatidylcholine, distearoylphosphatidylcholine, dilauroylphosphatidylcholine, or diarachidoylphosphatidylcholine.

89. A method of preparing lipospheres, the method comprising the steps of:
(a) providing at least one glyceride, at least one phospholipid, at least one compound and a dispersion medium;
(b) placing the glyceride, phospholipid, compound and dispersion medium in a reaction vessel and heating until all the ingredients have melted to form a mixture;
(c) homogenizing the mixture to form a dispersion; and
(d) cooling the mixture to a temperature from about 0° C. to about 10° C. while stirring the mixture until a suspension of lipospheres having an average particle size of at least about 3 microns and not greater than about 450 microns, is formed.

90. The method of claim 89 wherein step (a) further comprises providing at least one polymer.

91. The method of claim 90 wherein the polymer is a cellulose polymer.

92. The method of claim 91 wherein the polymer is carboxymethylcellulose salt, hydroxypropylmethylcellulose or methylcellulose.

93. The method of claim 89 further comprising the step of adding at least one polymer to the suspension of lipospheres formed in step (d).

94. The method of claim 93 wherein the polymer is a cellulose polymer.

95. The method of claim 94 wherein the polymer is carboxymethylcellulose salt, hydroxypropylmethylcellulose or methylcellulose.

96. The method of claim 89 wherein the compound is a drug.

97. The method of claim 96 wherein the drug contains bupivacaine.

98. The method of claim 89 wherein the glyceride is a monoglyceride, a diglyceride or a triglyceride.

99. The method of claim 98 wherein the glyceride is a triglyceride.

100. The method of claim 99 wherein the at least one triglyceride is tristearin, trilaurin, tricaprin, triarachidin or trimyristin.

101. The method of claim 89 wherein the phospholipid is a synthetic phospholipid.

102. The method of claim 101 wherein the at least one synthetic phospholipid is dimyristoylphosphatidylcholine, dipalmitoylphosphatidylcholine, distearoylphosphatidylcholine, dilauroylphosphatidylcholine, or diarachidoylphosphatidylcholine.

103. The method of claim 89 wherein the phospholipid is a natural phospholipid.

104. The method of claim 103 wherein the natural phospholipid is egg phosphatidylcholine or soy phosphatidylcholine.

105. A method of preparing lipospheres, the method comprising the steps of:
(a) providing at least one triglyceride, at least one phospholipid, at least one drug and a dispersion medium;
(b) placing the triglyceride, phospholipid, drug and dispersion medium in a reaction vessel and heating until all the ingredients have melted to form a mixture;
(c) homogenizing the mixture to form a dispersion; and
(d) cooling the mixture to a temperature from about 0° C. to about 10° C. while stirring the mixture until a suspension of lipospheres having an average particle size of at least about 3 microns and not greater than about 450 microns, is formed.

106. The method of claim 105 wherein step (a) further comprises providing at least one polymer.

107. The method of claim 106 wherein the polymer is gelatin, polyvinyl pyrrolidone, polyethylene glycol, alginate or a cellulose polymer.

108. The method of claim 107 wherein the cellulose polymer is carboxymethylcellulose salt, hydroxypropylmethylcellulose or methylcellulose.

109. The method of claim 105 further comprising the step of adding at least one polymer to the suspension of lipospheres formed in step (d).

110. The method of claim 109 wherein the polymer is gelatin, polyvinyl pyrrolidone, polyethylene glycol, alginate or a cellulose polymer.

111. The method of claim 110 wherein the cellulose polymer is carboxymethylcellulose salt, hydroxypropylmethylcellulose or methylcellulose.

112. The method of claim 105 wherein the drug contains bupivacaine.

113. The method of claim 105 wherein the at least one triglyceride is tristearin, trilaurin, tricaprin, triarachidin or trimyristin.

114. The method of claim 105 wherein the phospholipid is a synthetic phospholipid.

115. The method of claim 114 wherein the synthetic phospholipid is dimyristoylphosphatidylcholine, dipalmitoylphosphatidylcholine, distearoylphosphatidylcholine, dilauroylphosphatidylcholine, or diarachidoylphosphatidylcholine.

116. The method of claim 105 wherein the phospholipid is a natural phospholipid.

117. The method of claim 116 wherein the natural phospholipid is egg phosphatidylcholine or soy phosphatidylcholine.

118. A method of preparing lipospheres, the method comprising the steps of:
(a) providing at least one triglyceride, at least one phospholipid, at least one drug containing bupivacaine and a dispersion medium;
(b) placing the triglyceride, phospholipid, drug and dispersion medium in a reaction vessel and heating until all the ingredients have melted to form a mixture;
(c) homogenizing the mixture to form a dispersion; and
(d) cooling the mixture to a temperature from about 0° C. to about 10° C. while stirring the mixture until a suspension of lipospheres having an average particle size of at least about 3 microns and not greater than about 450 microns, is formed.

119. The method of claim 118 wherein step (a) further comprises providing at least one polymer.

120. The method of claim 119 wherein the polymer is gelatin, polyvinyl pyrrolidone, polyethylene glycol, alginate or a cellulose polymer.

121. The method of claim 120 wherein the cellulose polymer is carboxymethylcellulose salt, hydroxypropylmethylcellulose or methylcellulose.

122. The method of claim 118 further comprising the step of adding at least one polymer to the suspension of lipospheres formed in step (d).

123. The method of claim 122 wherein the polymer is gelatin, polyvinyl pyrrolidone, polyethylene glycol, alginate or a cellulose polymer.

124. The method of claim 123 wherein the cellulose polymer is carboxymethylcellulose salt, hydroxypropylmethylcellulose or methylcellulose.

125. The method of claim 118 wherein the at least one triglyceride is tristearin, trilaurin, tricaprin, triarachidin or trimyristin.

126. The method of claim 118 wherein the phospholipid is a synthetic phospholipid.

127. The method of claim 126 wherein the synthetic phospholipid is dimyristoylphosphatidylcholine, dipalmitoylphosphatidylcholine, distearoylphosphatidylcholine, dilauroylphosphatidylcholine, or diarachidoylphosphatidylcholine.

128. The method of claim 118 wherein the phospholipid is a natural phospholipid.

129. The method of claim 128 wherein the natural phospholipid is egg phosphatidylcholine or soy phosphatidylcholine.

130. A method of inducing or maintaining local anesthesia in a patient, the method comprising the steps of:
administering to a patient an effective amount of a pharmaceutical composition to induce local anesthesia, wherein said pharmaceutical composition comprises the pharmaceutical composition of claims 1, 8, 15, 24, 33, 42, 46, 50, 62, 68, 71, 74, 79 or 84.

131. The method of claim 130 further comprising a pharmaceutically acceptable excipient.
